(19) United States
(12) Patent Application Publication
(10) Pub. No.: US 2018/0199888 A1
(43) Pub. Date: Jul. 19, 2018

(54) COUNTING AND MONITORING RESPIRATORY RATE AND DETECTING PNEUMONIA IN CHILDREN

(71) Applicants: Sherin Varkey, Sana'a (YE); Hrishikesh Pradeep Divate, Pune (IN)

(72) Inventors: Sherin Varkey, Sana'a (YE); Hrishikesh Pradeep Divate, Pune (IN)

(21) Appl. No.: 15/872,681

(22) Filed: Jan. 16, 2018

Related U.S. Application Data
(60) Provisional application No. 62/446,772, filed on Jan. 16, 2017.

(51) Int. Cl. A61B 5/00; A61B 5/08

(52) U.S. Cl. CPC A61B 5/6898; A61B 5/0816; A61B 2560/0431; A61B 2562/227; A61B 5/7282

(57) ABSTRACT

A system and method for counting and monitoring respiratory rate and detecting pneumonia in children by way of a mobile medical device are disclosed. The device is supported by a mobile application that implements the method. The system can provide a cloud-based web service to enable medical professionals to confirm detected pneumonia in children from a distance while a non-medical professional tests respiration of the children and to detect the prevalence of pneumonia or other diseases in a given geographical area. The method counts and monitors respiratory rate of children according to guidelines for detecting pneumonia in children. A respiratory detection device provides respiratory evaluation data to the mobile application, which then detects whether the child has pneumonia based on the child's age and observed

### CLAIM OF BENEFIT TO PRIOR APPLICATION

This application claims benefit to U.S. Provisional Patent Application 62/446,772, entitled “MOBILE MEDICAL DEVICE, SYSTEM, AND METHOD FOR MONITORING RESPIRATORY RATE AND DETECTING PNEUMONIA IN CHILDREN,” filed Jan. 16, 2017. The U.S. Provisional Patent Application 62/446,772 is incorporated herein by reference.

### BACKGROUND

Embodiments of the invention described in this specification relate generally to detecting pneumonia, and more particularly, to a mobile medical device, system, and method for counting and monitoring respiratory rate to detect pneumonia in children.

Pneumonia accounts for 1.4 million deaths in children under-five years of age annually, representing eighteen percent (18%) of all annual under-five worldwide mortality. Sixty percent (60%) of these deaths occur in just ten countries in South Asia and Sub-Saharan Africa, the cause of which is often the delay in diagnosis or misdiagnosis. This is problematic because many of these deaths could be prevented if proper medical resources (including medical personnel and medical equipment and/or diagnostic devices) were readily available to treat pneumonia where it strikes children. However, many regions that are heavily impacted by childhood pneumonia lack sufficient medical resources, lack sufficient availability of existing medical resources, and/or are unable to transport existing medical resources to areas with insufficient and/or unavailable medical resources.

Existing mobile phone applications and devices are all based on manual observation of the breaths and merely function as timers or tally counters. Some devices connect directly to the child to acquire data from the patient. However, many children respond with fear, anxiety, or other reactive emotions when contact with their body occurs. Thus, both of the existing options are problematic—the first option being inexact due to the possibility of human error and the second option often resulting in reactive emotions, such as fear or anxiety.

Therefore, what is needed is a more mobile way to monitor respiration in children and determine whether the children have pneumonia without physically contacting the children.

### BRIEF DESCRIPTION

Some embodiments of the invention include a mobile medical device, a system, and a method for counting and monitoring respiratory rate and detecting pneumonia in children. In some embodiments, the mobile medical device is supported by a respiration detection device component and a mobile application for counting and monitoring respiratory rate and detecting pneumonia in children via the respiration detection device component. In some embodiments, the system provides a cloud-based web service to enable medical professionals to detect pneumonia in children from a distance while a non-medical professional operates the mobile medical device to count and monitor respiratory rate of the children. In some embodiments, the method for counting and monitoring respiratory rate and detecting pneumonia in children includes a process that counts and monitors respiratory rate of children according to guidelines for detecting pneumonia in children.

In some embodiments, the method is implemented in part as a mobile application that runs on a processor of the mobile medical device and reads respiration data received from the respiration detection device component. In some embodiments, the mobile application uses input parameters, such as the age of the child, to detect whether the child has pneumonia based on the observed respiratory rate.

### BRIEF DESCRIPTION OF THE DRAWINGS

Having thus described the invention in general terms, reference is now made to the accompanying drawings, which are not necessarily drawn to scale, and which show different views of different example embodiments, and wherein:

FIG. 1 conceptually illustrates an operator using a respiration detection device with a wired connection to a mobile medical device that is running a mobile application that counts and monitors respiratory rate to detect pneumonia in children.

FIG. 2 conceptually illustrates a process for using the respiration detection device and the mobile application running on the mobile medical device in some embodiments to count and monitor respiratory rate to detect pneumonia in children.

FIG. 3 conceptually illustrates a method for counting and monitoring respiratory rate and detecting pneumonia in children in some embodiments.

FIG. 4 conceptually illustrates a respiration detection device with a wireless connection to a mobile medical device that is running a mobile application that counts and monitors respiratory rate to detect pneumonia in children.

FIG. 5 conceptually illustrates an architecture of a child pneumonia detection system in some embodiments.

FIG. 6 conceptually illustrates an electronic system with which some embodiments of the invention are implemented.

### DETAILED DESCRIPTION

In the following detailed description of the invention, numerous details and examples of counting and monitoring respiratory rate to detect pneumonia in children are described. In describing embodiments of the invention, reference is made to certain trademarks and/or word marks, including Bluetooth® and Wi-Fi®. The Bluetooth® word mark and logos are registered trademarks owned by Bluetooth SIG, Inc. Wi-Fi® is a registered trademark of Wi-Fi Alliance. While reference is made to the products and technology associated with these trademarks and word marks, it will be clear and apparent to one skilled in the art that the scope of the invention is neither restricted by such products and technology nor to the embodiments set forth and that the invention can be adapted for use in any of several manners inclusive or exclusive of said products and technology.

As stated above, pneumonia accounts for too many childhood deaths in regions that lack sufficient medical infrastructure to treat every child. The problems that arise from the existing options are problematic—the first option being inexact due to the possibility of human error and the second option often resulting in reactive emotions, such as fear or anxiety. Embodiments described in this specification solve such problems by using a high accuracy respiratory rate monitor that measures and communicates a child's respiration rate to a mobile device application, which then uses the World Health Organization (WHO) guideline to detect pneumonia based on the child's age and measured respiratory rate.

Embodiments of the mobile medical device, system, and method for counting and monitoring respiratory rate and detecting pneumonia in children described in this specification differ from and improve upon currently existing options. In particular, some embodiments of the mobile medical device, system, and method for counting and monitoring respiratory rate and detecting pneumonia in children differ by providing automatic monitoring, detecting, and counting of a child's respiration, whereas in at least one of the previous mechanisms, community health workers (CHW) had to manually count a child's respiration rate, which is error-prone. One of the other existing pneumonia detection mechanisms relied on fear-inducing invasive/non-invasive medical device contacts with a child, which is error-prone due to the possibility of respiration fluctuations that result from adrenaline and other biochemical messaging that may occur when a child experiences fear, anxiety, or other reactive emotions. In particular, one of the commonly used existing mechanisms required specialized devices to be attached to a child to detect respiratory rate so as to determine whether the child has pneumonia. Not only is this prone to inaccurate respiration measurement, but increases the likelihood of passing infectious diseases between children, given the direct bodily contact required by this existing mechanism. In contrast, mobile medical device, system, and method for counting and monitoring respiratory rate and detecting pneumonia in children described in this specification uses automated and precise techniques to detect respiratory rates, and thereby detect pneumonia, without attaching anything to the child's body. This can also be used in hospital settings, such as pediatric and neonatal ICUs, for continuous real time monitoring of respiratory rates of children.

In addition, these embodiments improve upon the currently existing options because they were neither directed to precise detection of respiratory rates nor capable of automatically translating respiration measurements to WHO guidelines so as to detect pneumonia in children. By contrast, the mobile medical device is configured to use a respiration detection device component that detects and communicates respiratory rate to the application that implements the method of the present disclosure and runs on a processor of the mobile medical device. The application then uses input parameters such as the age of the child to detect whether the child has pneumonia based on the observed respiratory rate.

The mobile medical device, system, and method for counting and monitoring respiratory rate and detecting pneumonia in children of the present disclosure may be comprised of the following elements. This list of possible constituent elements is intended to be exemplary only and it is not intended that this list be used to limit the mobile medical device, system, and method of the present application to just these elements. Persons having ordinary skill in the art relevant to the present disclosure may understand there to be equivalent elements that may be substituted within the present disclosure without changing the essential function or operation of the mobile medical device, system, and method for counting and monitoring respiratory rate and detecting pneumonia in children.

1. A mobile computing device with a data communication and power interface (e.g., a mobile smartphone with a USB interface connection port or wireless support).

2. A respiration detection device component that is designed to detect respiratory rate and communicate the respiratory rate to the mobile computing device via a data communication and power connection (e.g., a sensor-based system on chip (SoC) module that can detect respiratory rate when directed at a human and that receives power from, sends data to, and receives data from the mobile computing device via USB cable connection, or alternatively, is battery operated and support wireless data transmissions to/from the mobile medical device, such as a Bluetooth connection).

3. A USB data/power cable and associated USB interface ports on the mobile computing device and the respiration detection device component (alternatively, a wireless Bluetooth connection between the mobile medical device and the respiration detection device is supported, with a battery powering the respiration detection device).

4. A mobile application that implements the method of the present disclosure and which runs on a processor of the mobile medical computing device, such that the mobile application is able to read and interpret the data coming in from the respiration detection device.

The various elements of the mobile medical device, system, and method for counting and monitoring respiratory rate and detecting pneumonia in children of the present disclosure may be related in the following exemplary fashion. It is not intended to limit the scope or nature of the relationships between the various elements and the following examples are presented as illustrative examples only. The respiration detection device component (2) connects to the mobile computing device (1) via the USB data/power cable (3). The mobile application (4) is installed on the mobile computing device (1) after which the mobile computing device is able to power the respiration detection device component (2) and send data to and receive (and read) data from the respiration detection device component.

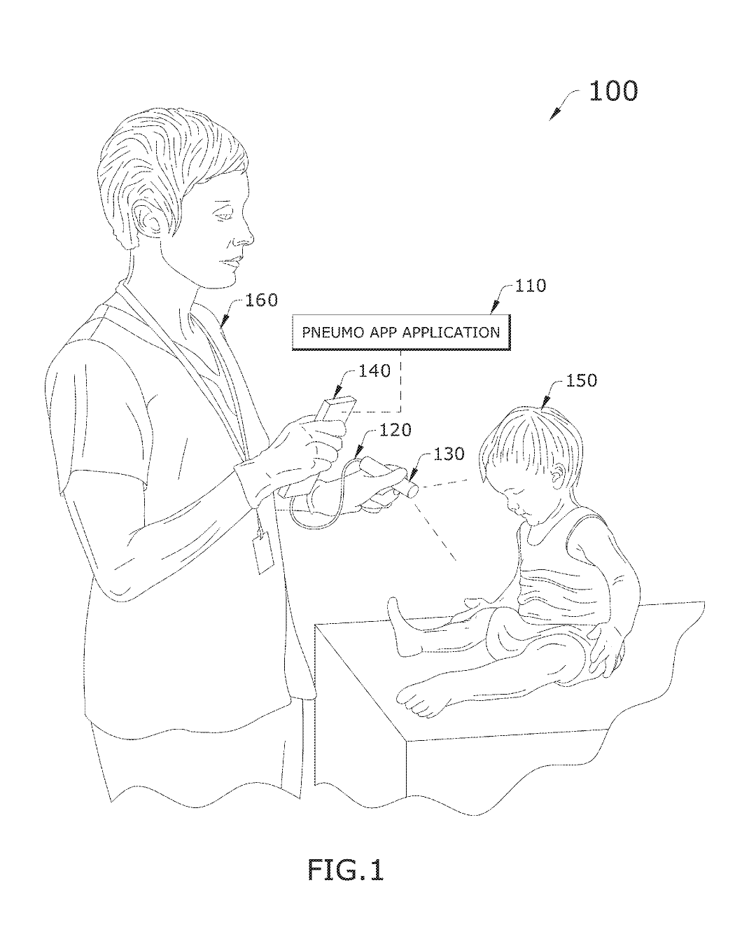

By way of example, FIG. 1 conceptually illustrates a pneumonia detection test of a child usage example 100. As shown in this figure, the pneumonia detection test of a child usage example 100 includes a “pneumo app” application 110 that runs on a mobile medical device 140 operated by a community health worker (CHW) 160 or other user. The CHW tests respiration of a child 150 by a respiration detection device 130 that has a wired USB cable 120 that provides connects the mobile medical device 140 to the respiration detection device 130 for transmission of data and/or power. By using the respiration detection device 130 in relation to the mobile medical device 140 with the pneumo app application 110 running, the CHW 160 effectively gather information and data related to counting and monitoring respiratory rate of the child 150 to determine whether the child 150 has pneumonia.

The mobile medical device, system, and method for counting and monitoring respiratory rate and detecting pneumonia in children of the present disclosure generally works by a user initiating a childhood pneumonia test. In doing so, the user may interact with the mobile computing device (via touch gesture on a touch sensitive display, or via another interface device) to open up the mobile application (4) on the mobile computing device. The user may select a preferred language (or simply use a previously determined language of choice) and input information about the child being test for possible pneumonia. The information that the user may input about the child includes at least the child's date of birth. Other information about the child may include, without limitation, and name of the child, parents or legal guardians of the child, etc.

The user would then position the respiration detection device component within a range of the child that would permit accurate measurement of the child's respiration. For instance, the respiration detection device component may be capable of accurate respiration detection and measurement if placed within one meter of a test subject (e.g., the child). While an example of one meter is herein described, a person skilled in the relevant art would appreciate that localized nuances may necessitate closer placement of the respiration detection device component in relation to the location of the child, whether the child is sitting, standing, or lying down (i.e., no restrictions on how the child is positioned at the location so long as the child is stationary). The user may then select an option to start the respiratory rate monitoring.

As the respiratory rate monitoring starts, the mobile application (4) repeatedly receives respiration data from the respiration detection device component and reads the respiratory rate of the child. After a few minutes (e.g., two minutes, two-and-a-half minutes, three minutes, or similar) of readings, the mobile application (4) calculates a statistical respiratory rate, such as the average or the maximum based on all the readings, and then compares the statistical respiratory rate to the known average respiratory rate for diagnosing pneumonia for a child in that particular age bracket. If it is equal to or exceeds that limit, then the mobile application (4) determines that the child has pneumonia. Upon determining that the child has pneumonia, the mobile application (4) may notify (e.g., by displaying an alert message) the user of the mobile computing device (1) or may include the pneumonia determination in a report that gets generated upon completion of the test. On the other hand, when all the repeated respiration readings are gathered but the statistical respiratory rate observed is less than the known average respiratory rate for diagnosing pneumonia in a child of a particular age, then the mobile application (4) determines that child does not have pneumonia, and may so inform the user of the mobile computing device (1).

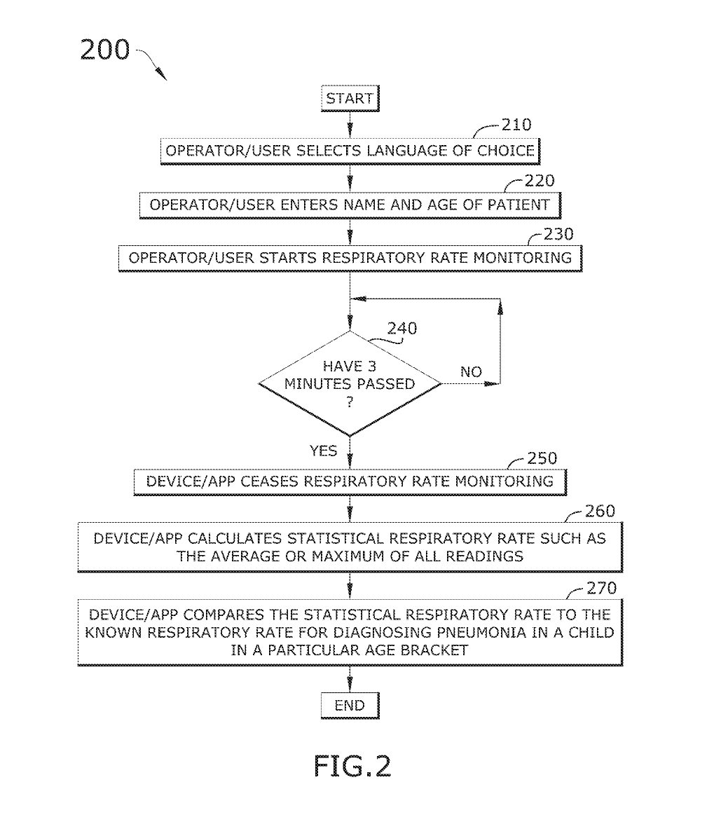

By way of example, FIG. 2 conceptually illustrates a process for using the respiration detection device 200 and the mobile application running on the mobile medical device to count and monitor respiratory rate to detect pneumonia in children. As shown in this figure, the process for using the respiration detection device 200 includes several steps 210-270. Specifically, an operator/user selects a language of choice (at 210) and enters a name and the date of birth of the patient (at 220). For example, the operator may be a community health worker in a remote location of a country and is testing respiration of children to determine whether any have pneumonia, and therefore, will enter the name and date of birth of each child, before performing respiration evaluation of the child.

During the next step of the process for using the respiration detection device 200, the operator/user starts the respiratory rate monitoring (at 230). For example, the operator/user starts the respiration detection device by way of the pneumo app running on the mobile medical device, while holding the respiration detection device proximate to the child being evaluated. Next, the process for using the respiration detection device 200 determines (at 240) whether three minutes have passed. In some embodiments, the respiration detection device obtains respiration count over a three minute period of time. When less then three minutes have elapsed since starting the respiration detection device for respiratory rate monitoring, then the process for using the respiration detection device 200 returns to the determination step (at 240). The process for using the respiration detection device 200 will continue to check whether three minutes have passed before moving forward.

In some embodiments, when the process 200 determines (at 240) that three minutes have passed, then the pneumo app running on the mobile medical device stops monitoring of the respiratory rate of the child (at 250). In some embodiments, the pneumo app sends a signal to the respiration detection device to stop functioning for respiration rate monitoring (e.g., by command transmitted via USB cable or wirelessly). Next, the pneumo app calculates a statistical respiratory rate based on respiratory rate monitoring data gathered during the three minute period. In some embodiments, the statistical respiratory rate calculated includes calculation of an average respiratory rate and/or identification of a maximum among all readings during the three minutes. After calculating the statistical respiratory rate, the pneumo app running on the mobile medical device compares the statistical respiratory rate to the known respiratory rate for diagnosing pneumonia in a child within a particular age bracket. Then the process for using the respiration detection device 200 ends.

In some embodiments, determining whether the child has pneumonia is based on the following conditions, which the pneumo app running on the mobile medical device calculates automatically: (i) the child is between zero (0) and fifty-nine (59) days old and their respiratory rate is sixty (60) or more breaths per minute, (ii) the child is between sixty (60) to three-hundred sixty-four (364) days old and the child's respiratory rate is fifty (50) or more breaths per minute, and (iii) the child is between three-hundred sixty-five (365) and one-thousand eight-hundred twenty-four (1824) days old and the child's respiratory rate is forty (40) or more breaths per minute.

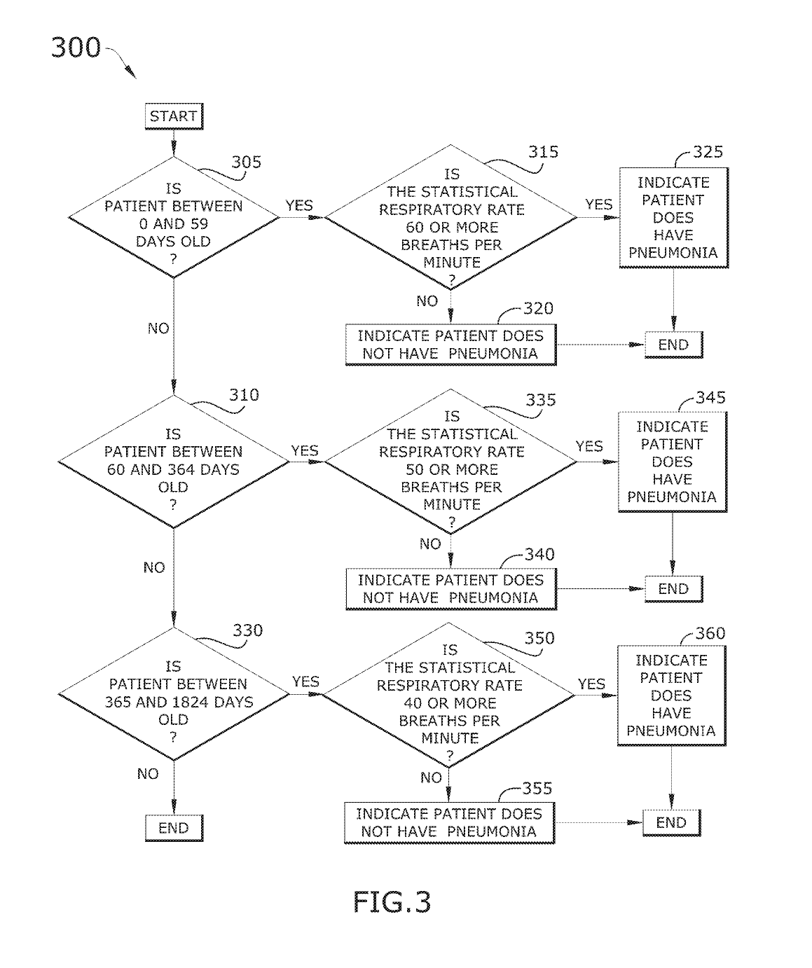

By way of example, FIG. 3 conceptually illustrates a method for counting and monitoring respiratory rate and detecting pneumonia in children 300. As shown in this figure, the method for counting and monitoring respiratory rate and detecting pneumonia in children 300 first determines (at 305) whether the patient (child) is between 0 and 59 days old (i.e., day 0 and day 59 included). When the patient is not between 0 and 59 days old, the method for counting and monitoring respiratory rate and detecting pneumonia in children 300 transitions to the next step to determine (at 310) whether the patient is within another age bracket (i.e., between 60 and 364 days old, inclusive), which is described in greater detail below.

On the other hand, when the patient is determined (at 305) to be between 0 and 59 days old (inclusive), then the method for counting and monitoring respiratory rate and detecting pneumonia in children 300 determines (at 315) whether the statistical respiratory rate is 60 or more breaths per minute. When the statistical respiratory rate is less than 60 breaths per minute, the method for counting and monitoring respiratory rate and detecting pneumonia in children 300 indicates (at 320) that the patient does not have pneumonia. Then the method for counting and monitoring respiratory rate and detecting pneumonia in children 300 ends. However, when the statistical respiratory rate is 60 or more breaths per minute, then the method for counting and monitoring respiratory rate and detecting pneumonia in children 300 indicates (at 325) that the patient does have pneumonia. Then the method for counting and monitoring respiratory rate and detecting pneumonia in children 300 ends.

In some embodiments, the method for counting and monitoring respiratory rate and detecting pneumonia in children 300 determines (at 310) whether the patient is between 60 and 364 days old (inclusive). When the patient is not between 60 and 364 days old, the method for counting and monitoring respiratory rate and detecting pneumonia in children 300 transitions to the next step to determine (at 330) whether the patient is within another age bracket (i.e., between 365 and 1824 days old, inclusive), described further below.

On the other hand, when the patient is determined (at 310) to be between 60 and 364 (inclusive) days old, then the method for counting and monitoring respiratory rate and detecting pneumonia in children 300 determines (at 335) whether the statistical respiratory rate is 50 or more breaths per minute. When the statistical respiratory rate is less than 50 breaths per minute, the method for counting and monitoring respiratory rate and detecting pneumonia in children 300 indicates (at 340) that the patient does not have pneumonia, and then ends. In contrast, when the statistical respiratory rate is at least 50 breaths per minute, then the method for counting and monitoring respiratory rate and detecting pneumonia in children 300 indicates (at 345) that the patient does have pneumonia, and then ends.

When the patient is not determined to be between 60 and 364 (inclusive) days old, the method for counting and monitoring respiratory rate and detecting pneumonia in children 300 determines (at 330) whether the patient is between 365 and 1824 days old (inclusive). When the patient is not between 365 and 1824 days old, the method for counting and monitoring respiratory rate and detecting pneumonia in children 300 ends. However, when the patient is between 365 and 1824 days old (including days 365 and 1824), then the method for counting and monitoring respiratory rate and detecting pneumonia in children 300 determines (at 350) whether the statistical respiratory rate is 40 or more breaths per minute. When the statistical respiratory rate is less than 40 breaths per minute, the method for counting and monitoring respiratory rate and detecting pneumonia in children 300 indicates (at 355) that the patient does not have pneumonia, and then ends. However, when the statistical respiratory rate is at least 40 breaths per minute, then the method for counting and monitoring respiratory rate and detecting pneumonia in children 300 indicates (at 360) that the patient does have pneumonia, and then ends.

To make the mobile medical device, system, and method for counting and monitoring respiratory rate and detecting pneumonia in children of the present disclosure, one may procure a respiration detection device component mentioned above and develop a mobile application specifically to communicate with it and use the relevant logic described above.

To use the mobile medical device, system, and method for counting and monitoring respiratory rate and detecting pneumonia in children of the present disclosure, a user, such as community health worker (CHW), may identify children suspected of having pneumonia and test them. Specifically, because the mobile medical device, system, and method for counting and monitoring respiratory rate and detecting pneumonia in children could be used to monitor respiratory rates of patients where there is no electricity or in remote areas where there are limited medical capabilities, the CHW may travel to rural areas to help diagnose health problems and, upon finding a child who is suspected of having contracted pneumonia, may assemble the respiration detection device component in connection with the mobile computing device, and thereafter open up the mobile application and start the method for detecting pneumonia. If pneumonia is detected, the CHW may note the affected location and family location and may either refer them to a doctor for treatment of pneumonia as needed or treat the child herself/himself depending upon the capacity of the CHW and the existing pneumonia management guidelines in the country.

Additional features of the mobile medical device, system, and method for counting and monitoring respiratory rate and detecting pneumonia in children described in this specification include the capability of routine and critical care monitoring of neonates and pediatric age children, data persistence of captured respiration data (e.g., only local storage unit of mobile medical computing device and/or in a cloud database when connected to a cloud service), report generation (e.g., specific data points related to a child's measured respiration, such as average respiration rate, minimum respiration rate, maximum respiration rate, etc.) contemporaneous video recording while detecting and measuring respiration (e.g., a video recording of the child whose respiration is being measured during a typical three minute respiration test time), video recording persistence (e.g., local storage and/or cloud database), language support in any of several languages and support of audible command input (especially important in areas of relatively low literacy), location awareness by receiving location data of mobile medical computing device from global positioning system (GPS) satellites, as well as persistent storage of location data in connection with stored respiratory data of children and/or used in connection with various reports and so on).

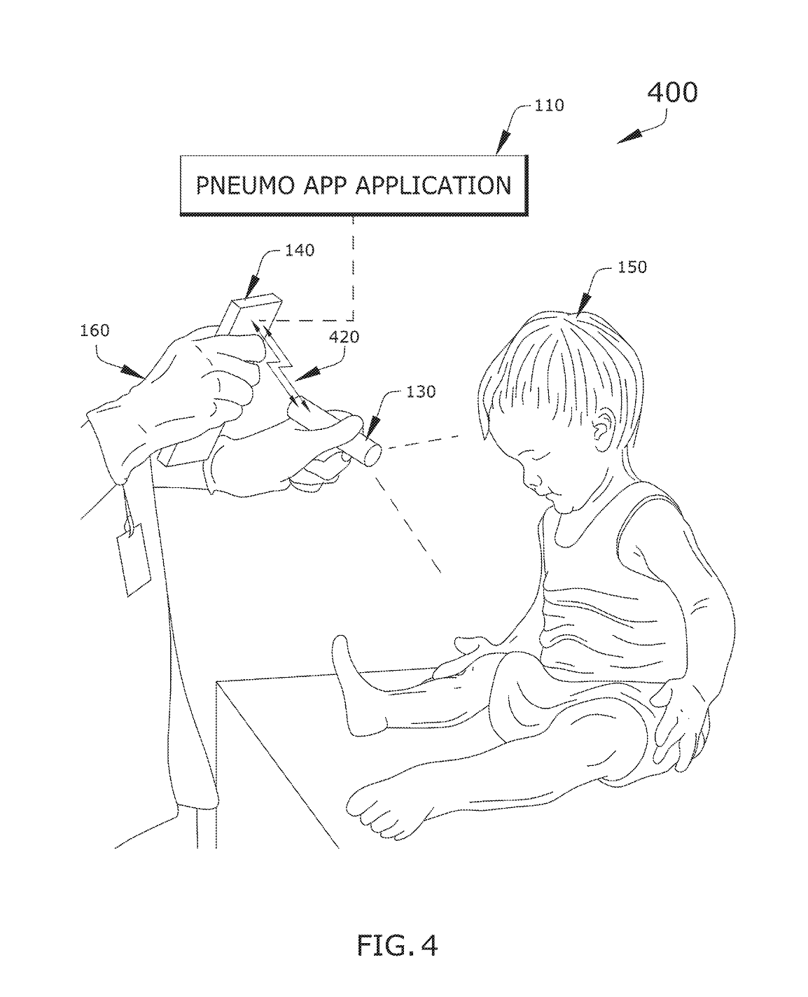

In some embodiments, the mobile medical device and application can facilitate wired or wireless connection to the respiration detection device. An example of a wireless connection would be a Bluetooth connection between a respiration detection device and mobile medical device. In some cases, a single community health worker or other user can operate both the mobile medical device and the respiration detection device, while it is also possible for one user to operate the mobile medical device with another user holding/operating the respiration detection device nearby a child. By way of example, FIG. 4 conceptually illustrates a child pneumonia detection example 400 of an operator 160 with a mobile medical device 140 that is running a pneumo app application 110 in relation to a respiration test of a child 150 in which a respiration detection device 130 counts and monitors respiration of the child 150 and transmits respiration data to the mobile medical device 140 by way of a wireless connection 420.

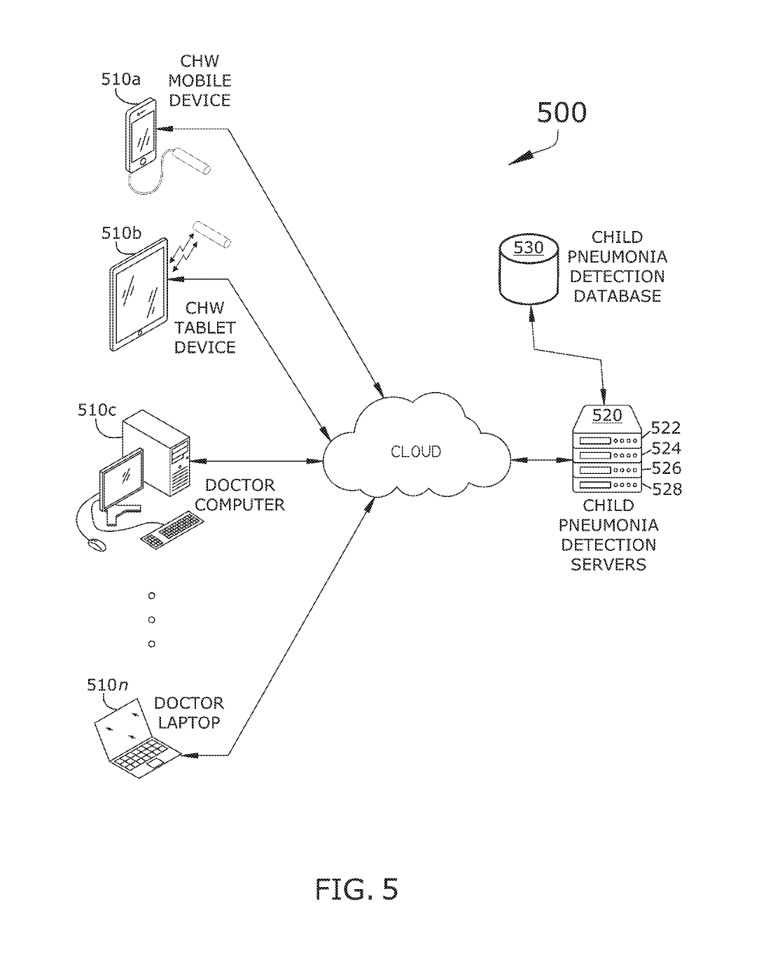

Now turning to another embodiment, FIG. 5 conceptually illustrates an architecture of a child pneumonia detection system 500. As shown in this figure, the child pneumonia detection system 500 includes a plurality of mobile medical devices 510a-510b, a plurality of computing devices 510c-510n, a set of child pneumonia detection servers 520, and child pneumonia detection database 530. The set of child pneumonia detection servers 520 includes at least one child pneumonia detection web service host computing device 528, which provides a hosted connection network address (e.g., an IP address, a web URL, etc.) to which the plurality of mobile medical devices 510a-510b and the plurality of computing devices 510c-510n can connect to from any location at which a network connection can be established in order to access the child pneumonia detection system 500. The set of child pneumonia detection servers 520 may also include separate computing device servers, such as (i) a user authentication server 526, which performs a set of authentication operations in relation to users or operators (such as doctors or community health workers) trying to access the child pneumonia detection system 500, (ii) a database management server 522 that interfaces with the child pneumonia detection database 530 to store and retrieve information related to respiratory data and/or evaluations of children, and/or (iii) an authorized user server computing device 524 that registers each new user of the child pneumonia detection system 500 (e.g., when a doctor or an authorized community health worker registers).

Each of the mobile medical devices 510a-510b and computing devices 510c-510n connects to the child pneumonia detection servers 520 over a network (labeled “cloud” in the child pneumonia detection system 500 shown in this figure), such as the Internet, to send and receive data in relation to respiration tests of children. The mobile medical device 510a is operated by a community health worker (CHW) and includes a smartphone mobile device and a wired respiration detection device. The mobile medical device 510b is also operated by a CHW and includes a tablet computing device and a wireless respiration detection device. The wireless respiration detection device may communicate wirelessly with the tablet computing device, e.g., over Bluetooth. In some embodiments, an application running on the mobile medical device operated by the CHW aggregates all of the data from the respiration detection device and information about each child, and then transmits the data and information over the cloud to the child pneumonia detection servers 520. In some embodiments, respiration test data and information for each child is temporarily stored on the mobile medical devices 510a-510b, when the CHW is administering the respiration test at a location with no network coverage. In such cases, the application running on the mobile medical device can upload the data later, when a network connection to the child pneumonia detection system 500 can be made.

In contrast to the mobile medical devices 510a-510b, each of the computing devices 510c-510n receives data in relation to child respiration tests performed by operators (e.g., community health workers) of the mobile medical devices. Specifically, each of the computing devices 510c-510n is operated by a doctor or another authorized health professional. When respiration test data and information is received, the doctor (or other health professional) can evaluate the data and make determinations as to whether or not the child has pneumonia. However, because the pneumo app running on the mobile medical device provides a clear indication of whether pneumonia is detected, the doctor (or other health professional) who receives the respiration test data and information via the cloud-based network system serves a purpose of confirming what the CHW has seen (confirming whether or not pneumonia is detected). The cloud-based network system also enables recording, aggregating, and monitoring of the prevalence of pneumonia or other diseases in a given geographical area. In some embodiments, the confirmed determination of the doctor or health professional is uploaded to the child pneumonia detection servers 520 for storage in the child pneumonia detection database 530 and for re-transmission to the corresponding mobile medical device of the CHW, thereby confirming for the CHW whether or not pneumonia is detected.

In some embodiments, the child pneumonia detection servers 520 can scale to accommodate multiple simultaneous mobile medical device and computing device connections. In some embodiments, the child pneumonia detection web service host computing device 528 along with the user authentication server 526 provide a login interface so that a user of a mobile medical device or computing device can access the child pneumonia detection system 500 through an existing registered account. In addition, in some embodiments, the child pneumonia detection web service host computing device 528 along with the user authentication server 526 performs authentication operations in relation to login attempts received from the mobile medical devices 510a-510b or the computing devices 510c-510n. In some embodiments, one or both of the login interface and the authentication operations are provided by or performed independently on the CHW's mobile medical device by the pneumo app running.

Many of the above-described features and applications are implemented as software processes that are specified as a set of instructions recorded on a computer readable storage medium (also referred to as computer readable medium or machine readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

In this specification, the term “software” is meant to include firmware residing in read-only memory or applications stored in magnetic storage, which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

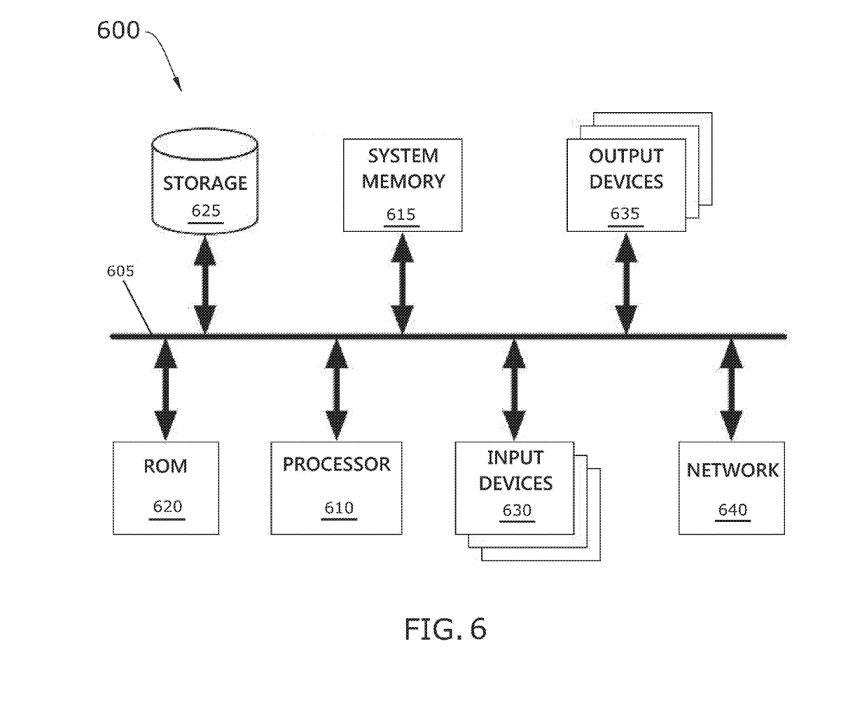

FIG. 6 conceptually illustrates an electronic system 600 with which some embodiments of the invention are implemented. The electronic system 600 may be a computer, phone, PDA, or any other sort of electronic device. Such an electronic system includes various types of computer readable media and interfaces for various other types of computer readable media. Electronic system 600 includes a bus 605, processing unit(s) 610, a system memory 615, a read-only 620, a permanent storage device 625, input devices 630, output devices 635, and a network 640.

The bus 605 collectively represents all system, peripheral, and chipset buses that communicatively connect the numerous internal devices of the electronic system 600. For instance, the bus 605 communicatively connects the processing unit(s) 610 with the read-only 620, the system memory 615, and the permanent storage device 625.

From these various memory units, the processing unit(s) 610 retrieves instructions to execute and data to process in order to execute the processes of the invention. The processing unit(s) may be a single processor or a multi-core processor in different embodiments.

The read-only-memory (ROM) 620 stores static data and instructions that are needed by the processing unit(s) 610 and other modules of the electronic system. The permanent storage device 625, on the other hand, is a read-and-write memory device. This device is a non-volatile memory unit that stores instructions and data even when the electronic system 600 is off. Some embodiments of the invention use a mass-storage device (such as a magnetic or optical disk and its corresponding disk drive) as the permanent storage device 625.

Other embodiments use a removable storage device (such as a floppy disk or a flash drive) as the permanent storage device 625. Like the permanent storage device 625, the system memory 615 is a read-and-write memory device. However, unlike storage device 625, the system memory 615 is a volatile read-and-write memory, such as a random access memory. The system memory 615 stores some of the instructions and data that the processor needs at runtime. In some embodiments, the invention's processes are stored in the system memory 615, the permanent storage device 625, and/or the read-only 620. For example, the various memory units include instructions for processing appearance alterations of displayable characters in accordance with some embodiments. From these various memory units, the processing unit(s) 610 retrieves instructions to execute and data to process in order to execute the processes of some embodiments.

The bus 605 also connects to the input and output devices 630 and 635. The input devices enable the user to communicate information and select commands to the electronic system. The input devices 630 include alphanumeric keyboards and pointing or cursor control devices. The output devices 635 display images generated by the electronic system 600. The output devices 635 include printers and display devices, such as cathode ray tubes (CRT) or liquid crystal displays (LCD). Some embodiments include a touchscreen that functions as both an input and output device.

Finally, as shown in FIG. 6, bus 605 also couples electronic system 600 to a network 640 through a network adapter (not shown). In this manner, the computer can be a part of a network of computers (such as a local area network (“LAN”), a wide area network (“WAN”), or an Intranet), or a network of networks (such as the Internet). Any or all components of electronic system 600 may be used in conjunction with the invention.

These functions described above can be implemented in digital electronic circuitry, in computer software, firmware or hardware. The techniques can be implemented using one or more computer program products. Programmable processors and computers can be packaged or included in mobile devices. The processes and logic flows may be performed by one or more programmable processors and by sets of programmable logic circuitry. General and special purpose computing and storage devices can be interconnected through communication networks.

Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD−RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the invention has been described with reference to numerous specific details, one of ordinary skill in the art will recognize that the invention can be embodied in other specific forms without departing from the spirit of the invention. For instance, FIGS. 2 and 3 conceptually illustrate processes in which the specific operations of these processes may not be performed in the exact order shown and described. Specific operations may not be performed in one continuous series of operations, and different specific operations may be performed in different embodiments. Furthermore, the processes could be implemented using several sub-processes, or as part of a larger macro process. Thus, one of ordinary skill in the art would understand that the invention is not to be limited by the foregoing illustrative details, but rather is to be defined by the appended claims.

I claim:

1. A mobile medical device that counts and monitors respiratory rate to detect pneumonia in children comprising:
a mobile computing device comprising a processor and a first USB data/power interface port;
a respiration detection device component comprising a second USB data/power interface port, wherein the respiration detection device component is designed to detect respiratory rate of a child and transmit the respiratory rate to the mobile computing device via the second USB data/power interface port;
a USB data/power cable that connects via a first USB connector to the first USB data/power interface port of the mobile computing device and connects via a second USB connector to the second USB data/power interface port of the respiration detection device component; and
a mobile application that runs on the processor of the mobile computing device, such that the mobile application is able to read and interpret the data coming in from the respiration detection device component.
